(19) United States
(12) Patent Application Publication
(10) Pub. No.: US 2004/0234962 A1
(43) Pub. Date: Nov. 25, 2004

(54) Multicoated or multilayer entrapment matrix for protein biosensor

(76) Inventors: Javier Alarcon, Durham, NC (US); Helen V. Hsieh, Durham, NC (US); Jon A. Rowley, Chapel Hill, NC (US); Ross W. Jacobson, Durham, NC (US); J. Bruce Pitner, Durham, NC (US); Douglas B. Sherman, Durham, NC (US)

Correspondence Address: DAVID W. HIGHET, VP AND CHIEF IP COUNSEL, BECTON, DICKINSON AND COMPANY, 1 BECTON DRIVE, MC 110, FRANKLIN LAKES, NJ, 07417-1880, US

(21) Appl. No.: 10/428,295

(22) Filed: May 2, 2003

(51) Int. Cl. C12Q 1/68

(52) U.S. Cl. 435/6

(57) ABSTRACT

Multicoated or multilayer matrices capable of embedding, encapsulating or entrapping binding proteins, optionally with a reporter group attached, specific for analytes of interest, in particular sugars, and methods of making and using such matrices.

### BACKGROUND OF THE INVENTION

1. Field of the invention

The invention is directed to multicoated or multilayer matrices capable of entrapping binding proteins specific for analytes of interest, and methods of making and using such matrices.

2. Background Information

Monitoring in vivo concentrations of physiologically relevant compounds to improve diagnosis and treatment of various diseases and disorders is a desirable goal and would enhance the lives of many individuals. Advances in this area show particular promise in the area of facilitating adequate metabolic control in diabetics. Currently, most diabetics use the “finger stick” method to monitor blood glucose level, and patient compliance is problematic due to pain caused by frequent (several times per day) sticks. As a consequence, there have been efforts to develop non-invasive or minimally invasive in vivo and more efficient in vitro methods for frequent and/or continuous monitoring of blood glucose or other glucose-containing biological fluids.

Some of the most promising methods for monitoring in vivo concentrations of physiologically relevant compounds involve the use of a biosensor. Biosensors are devices capable of providing specific quantitative or semi-quantitative analytical information using a biological recognition element that is combined with a transducing (detecting) element.

To develop reagentless, self-contained, and/or implantable and/or continuous biosensors using proteins, the transduction element must be in communication with a detection device to interrogate the signal to and from the transduction element. Typical methods include placing proteins within or onto the surface of optical fibers or planar waveguides using immobilization strategies. Such immobilization strategies include, but are not limited to, entrapment of the protein within semi-permeable membranes, organic polymer matrices, or inorganic polymer matrices. The immobilization strategy used may ultimately determine the performance of the working biosensor. The prior art details numerous problems associated with the immobilization of biological molecules. For example, many proteins undergo irreversible conformational changes, denaturation, and loss of biochemical activity. Immobilized proteins can exist in a large number of possible orientations on any particular surface, for example, with some proteins oriented such that their active sites are exposed and others oriented such that their active sites are not exposed (and thus not able to undergo selective binding reactions with the analyte). Immobilized proteins are also subject to time-dependent denaturation, denaturation during immobilization, and leaching of the entrapped protein subsequent to immobilization. This results in problems including, for example, an inability to maintain calibration of the sensing device and signal drift. Immobilization of proteins that have been modified with extrinsic dyes or reporter groups presents further challenges as the immobilization method must not interfere with the reporter group function. In general, binding proteins require orientational control and conformational freedom to enable effective use, thus many physical absorption and random or bulk covalent surface attachment or immobilization strategies as taught in the literature generally are either suboptimal or unsuccessful.

There have been several reports of encapsulating proteins and other biological systems into simple inorganic silicon matrices formed by low temperature sol-gel processing methods (e.g., Brennan, J. D. Journal of Fluorescence 1999, 9(4), 295-312, and Flora et al., Analytical Chemistry 1998, 70 (21), 4505-4513). In order to be functional, entrapped or immobilized binding proteins must remain able to undergo at least some analyte-induced conformational change. It has been reported that sol-gel entrapped proteins can exhibit dramatically altered binding constants, or binding constants that change over relatively short time periods or under varying environmental conditions (Brennan, 1999). In addition, the activity of proteins entrapped in sol-gel matrices has been reported to be time dependent, a characteristic that limits general applicability of sol-gels in biosensors for in vitro as well as in vivo use. Nanoporous TiO₂ films have also been used to entrap binding proteins but suffer from many of the same problems as silicon-derived sol-gels (Topoglidis, et al., Analytical Chemistry 1998, 70,5111-5113)

Therefore, there is a need in the art to design improved analyte-permeable matrices, wherein binding proteins specific for particular analytes can be embedded, entrapped or encapsulated, for interfacing to signal transmitting and receiving elements.

### SUMMARY OF THE INVENTION

The present invention provides a multicoated or multilayer matrix in which a binding protein specific for an analyte of interest may be embedded, entrapped or encapsulated, optionally with a reporter group, such that a real time measure of analyte concentration may be obtained, for example, by measuring the fluorescence of a reporter group. The multicoated or multilayer matrix preferably comprises three coatings or layers: (i) a core in which a binding protein may be physically or covalently embedded, entrapped or encapsulated; (ii) a containment layer or coating that surrounds the core and ensures its integrity; and (iii) an outer layer that provides selectivity of the matrix to the analyte of interest. The invention also provides an optional fourth layer or coating that may be used to make the matrix biocompatible. Some layers or coating compositions may combine one or more of these properties, for example a layer may provide both biocompatibility and selectivity or may provide both containment and selectivity properties, thereby eliminating the need for a unique individual layer with the desired property.

The core comprises a composition comprising an embedding, encapsulating or entrapment compound such as alginate or chitosan, or other suitable polymers. The containment coating or layer comprises a composition such as poly-lysine, low and high molecular weight polyvinyl alcohols (PVAs), N-methyl-4(4′-formylstyryl) pyridinium methosulfate acetal (SbQ-PVA), Nafion®, or polyurethanes. The outer layer or coating comprises a polymer coating permeable to the analyte of interest such as SbQ-PVA, hydroxyethyl methacrylate (HEMA), PVA, alginate, polyurethanes, or inorganic, or organically modified inorganic sol-gels.

The invention also provides a biosensor that is suitable for measuring the concentration of an analyte of interest in vivo or in vitro. The biosensor of the present invention comprises a multilayered or multicoated matrix with at least one binding protein specific for an analyte of interest, optionally, with at least one reporter group associated therewith or attached thereto that is capable of determining the concentration of the analyte of interest. In one preferred embodiment, the analyte measured is glucose and/or related sugars.

The invention further provides a device for measuring glucose concentrations that is suitable for in vivo use comprising a mutated glucose/galactose binding protein that is embedded, entrapped or encapsulated within a matrix that is permeable to analytes of interest and, optionally, at least one reporter group attached to the binding protein such that the reporter group provides a detectable and reversible signal when the mutated glucose/galactose binding protein is exposed to varying glucose concentrations.

In addition, the invention provides a method of measuring the concentration of an analyte of interest comprising: i) contacting the multicoated or multilayer matrix containing the binding protein with, optionally, at least one reporter group attached thereto, with a solution, either in vivo or in vitro; ii) exposing the matrix to an energy source capable of producing a signal; and iii) measuring the signal emitted by the binding protein or reporter group associated therewith. The intensity of the signal is directly correlated with the amount of analyte present in the solution. Preferably, the analyte of interest is glucose or related sugars and the binding protein is the mutated glucose/galactose binding protein.

In another aspect, the invention provides a method of making a multicoated or multilayer matrix comprising the steps of: (a) forming a core; (b) contacting the core with a first coating solution for a sufficient time to coat or layered the core; (c) isolating and drying the first coating on the core to form the containment layer or coating; (d) contacting the containment layer or coating with a second coating solution to form a multi-coated three dimensional structure; and (e) isolating and drying the second coating on the multi-coated three dimensional structure to obtain a multicoated or multilayered matrix. Preferably, a binding protein specific for an analyte of interest is embedded, entrapped or encapsulated in the core. Optionally, the binding protein may be covalently attached to the core of the matrix.

### BRIEF DESCRIPTION OF THE DRAWINGS

FIG. 1 illustrates the structure of a matrix according to one embodiment of the invention.

FIG. 2 shows the effect of the outermost layer on leaching of binding protein from a matrix embodiment of the instant invention.

FIG. 3 shows effects on I/Io of different polymers used as a second layer on a core matrix of binding, protein entrapped in alginate.

FIG. 4 demonstrates fluorescence response of a three-layer matrix biosensor measuring glucose in rabbit serum and blood.

FIG. 5 illustrates directed labeling of binding protein by ligand masking.

FIG. 6 demonstrates binding protein and binding protein-hydrogel polymer conjugate fluorescence emission in the presence (+) and absence (−) of glucose.

FIG. 7 shows glucose titration and dissociation constants for binding protein and binding protein-hydrogel polymer conjugate.

### DETAILED DESCRIPTION OF THE INVENTION

The following detailed description of the invention is not intended to be illustrative of all embodiments. In describing preferred embodiments of the present invention, specific terminology is employed for the sake of clarity. However, the invention is not intended to be limited to the specific terminology so selected. It is to be understood that each specific element includes all technical equivalents that operate in a similar manner to accomplish a similar purpose.

The present invention provides a matrix, either multicoated or multilayered, in which a binding protein specific for an analyte of interest may be embedded, entrapped or encapsulated, optionally with a reporter group, such that a real time measure of analyte concentration may be obtained.

As used herein, “matrix” refers to an essentially three-dimensional environment capable of immobilizing, by embedding, entrapping or encapsulating at least one binding protein for the purpose of measuring a detectable signal corresponding to one or more analyte concentrations. The relationship between the constituents of the matrix and the binding protein include, but are not limited to, covalent, ionic, and van der Waals interactions and combinations thereof. The matrix provides for a binding protein transducing element configuration that may, for example, be incorporated at the distal end of a fiber or other small minimally invasive probe to be inserted within the tissue of a patient, to enable an episodic, continuous, or programmed reading to the patient or health care provider. Information from the transducing element to the patient or health care provider may be provided, for example, by telemetry, visual, audio, or other means known in the art, for example, as taught in U.S. Pat. No. 5,517,313, U.S. Pat. No. 5,910,661, U.S. Pat. No. 5,894,351, and U.S. Pat. No. 5,342,789 as well as in Beach et al., IEEE Transactions on Instrumentation and Measurement 1999, 48(6) 1239-1245. Information includes electrical, mechanical, and actinic, radiation suitable for deriving analyte concentration or change in concentration, as is suitable.

Numerous hydrogels may be used in the present invention for one or more of the matrix layers. The hydrogels may be, for example, polysaccharides such as agarose, dextran, carrageenan, alginic acid, starch, cellulose, or derivatives of these such as, e.g., carboxymethyl derivatives, or water-swellable organic polymers such as, e.g., polyvinyl alcohol, polyacrylic acid, polyacrylamide, polyethylene glycol, copolymers of styrene and maleic anhydride, polyurethanes, copolymers of vinyl ether and maleic anhydride and derivatives thereof. Derivatives providing for covalently crosslinked networks are preferred. Synthesis and biomedical and pharmaceutical applications of hydrogels comprising polypeptides are known in the art. (See, e.g. “Biosensors Fundamentals and Applications”, edited by A. D. F. Turner, I. Karube and G. S. Wilson; published from Oxford University Press, in 1988). An exemplary hydrogel matrix derived from a water-soluble, ultraviolet (UV) crosslinkable polymer from the class of cationic stibazolium hemicyanines comprises poly(vinyl alcohol), N-methyl-4(4′-formylstyryl)pyridinium methosulphate acetal (CAS Reg. No. [107845-59-0]) available from PolyScience Warrington, Pa. Thiol-reactive hydrogel polymers can be readily derived from materials with free carboxylate groups such as carboxymethyl cellulose (CMC) or polyacrylic acid. Other polymers with thiol-reactive groups may also be used, for example, high molecular weight polethylene glycol (PEG) with thiol-reactive maleimide groups, which is available commercially (Shearwater Corp., Huntsville, Ala.).

The polymer portion of the hydrogel may contain one or more functionalities that are suitable for hydrogen bonding or covalent coupling (e.g. hydroxyl groups, amino groups, ether linkages, carboxylic acids and esters and the like) to either the protein or reporter group.

In one embodiment of the encapsulation process, one or more hydrogels in water is added to the mutated binding protein in an aqueous buffer solution having a pH in the range of about 4 to about 10 depending on the protein. Subsequent curing of the matrix, for example, crosslinking, provides physical form. Using this technique and a conventional fabrication process (e.g. block casting, reverse emulsion polymerization, screen or contact printing, fluid-bed coating and dip or spin coating) one can obtain matrices in various configurations (e.g. beads, granulates, nanoparticles, microparticles, monoliths, and thick and thin films) suitable for in vitro and in vivo use.

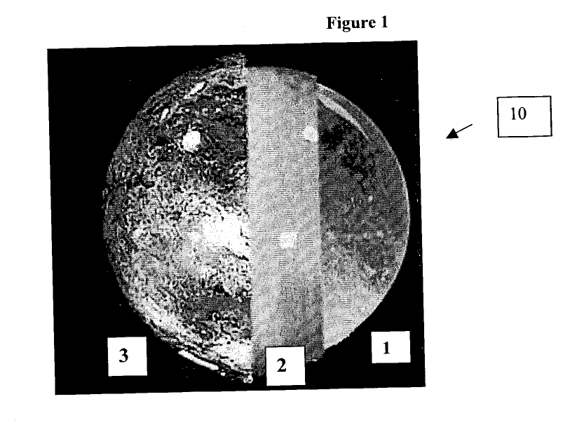

FIG. 1 illustrates the matrix of the instant invention. Multicoated particle 10, comprising a core 1, containment layer 2, and outer layer 3 is shown. The core enables entrapping, embedding, or encapsulating of a binding protein specific for an analyte of interest. In general, the core of the matrix is comprised of any suitable natural or synthetic polymer, such as a hydrogel, for example alginate or hyaluronate. In a preferred embodiment, the core 1 is alginate. In a particularly preferred embodiment, a binding protein, optionally with at least one reporter group selected to provide a signal upon binding with the analyte of interest, is covalently entrapped, embedded, or encapsulated within the core.

The embedding, encapsulating or entrapping material is used to form the core of the matrix. Preferably, the encapsulating core is a hydrogel, particularly alginate and hyaluronate, or another water-soluble, crosslinkable polymer including, but not limited to, cellulose acetate, pectin, chitosan and polymers derived from cellulose, like cellulose ethers. In one embodiment, the core is comprised of a compound that is suitable for covalent attachment of a binding protein. When the core comprises alginate, cross-linking may be with calcium ions; however, barium, strontium, and magnesium may also be used. If the binding protein is not covalently bound to the core, the core should be sufficiently cross-linkable to physically embed, entrap or encapsulate a binding protein. Determining the level of sufficient cross-linking of the material to avoid release of the protein is within the skill of one practiced in the art by routine experimentation.

In the case of covalent attachment of the protein to the matrix, it is desirable to first covalently attach the protein to the matrix, followed by crosslinking. However, crosslinking or partial crosslinking of the matrix followed by or simultaneously in combination with covalent attachment of protein is within the scope of the instant invention.

The containment layer 2 of the multicoated or multilayer matrix serves to contain the core and/or its contents, preserving its integrity, for example by preventing contact and/or interaction with the outer layer or with components of the external solution that may result in undesirable reactions (e.g. dissolution or degradation). Containment layer 2 may also provide selective barrier properties to prevent penetration by molecules based on their molecular weight or charge. The containment layer 2 may be comprised, for example, of poly-L-lysine, poly-D-lysine, poly-L-ornithine, low and high molecular weight polyvinyl alcohols (PVAs), cationic stilbazolium hemicyanine modified polyvinyl alcohols such as poly (vinyl alcohol), N-methyl-4(4′-fornylstyryl)pyridinium methosulfate acetal (SbQ-PVA), and perfluoroinated ion exchange copolymers such as Nafion® (tetrafluoroethylene and perfluoro-[2-(fluorosulfonylethoxy)propylvinyl ether] copolymer). Poly-L-lysine is particularly suitable.

The outer layer 3 of the multicoated or multilayer matrix serves to selectively allow the analyte of interest to penetrate the matrix and to contact or interact with the binding protein in the core, while preventing leaching of entrapped or encapsulated protein from the interior of the matrix. Preferably, the outer layer 3 of the multicoated or multilayer matrix is prepared from biocompatible materials or incorporates materials capable of minimizing adverse reactions with the body. Adverse reactions from implants include, inter alia, inflammation, protein fouling, tissue necrosis, immune response and leaching of toxic materials. Such materials or treatments are well known and practiced in the art, for example, as taught by Quinn, et al., Biomaterials 1995, 16(5), 389-396, and Quinn, et al., Biomaterials 1997, 18(24), 1665-1670. The outer layer 3 may be comprised, for example, of cross-linkable polymers or polymer precursors such as poly (vinyl alcohol), N-methyl-4(4′-formylstyryl)pyridinium methosulfate acetal (SbQ-PVA), poly (2-hydroxyethyl methacrylate) (pHEMA) and the like, alginate, or inorganic sol-gels modified with organic or inorganic reagents. In one particularly preferred embodiment, the outer layer 3 is comprised of inorganic sol-gels of silicon or titanium modified with organic or inorganic reagents.

Sol-gels useful for the present invention include material prepared by conventional, well-known sol-gel methods and include inorganic material, organic material or mixed organic/inorganic material. The materials used to produce the sol-gel can include, but are not limited to, aluminates, aluminosilicates and titanates. These materials may be augmented with the organically modified silicates, (osmosis) and functionalized siloxanes, to provide an avenue for imparting and manipulating hydrophilicity and hydrophobicity, ionic charge, covalent attachment of protein, and the like. As used herein the term “hydrolytically condensable siloxane” refers to sol-gel precursors having a total of four substituents, at least one, preferably two, and most preferably three or four of the substituents being alkoxy substituents covalently bound to silicone through oxygen and mixtures thereof. In the case of three, two, and one alkoxy substituent precursors, at least one of the remaining substituents preferably is covalently bound to silicone through carbon, and the remaining substitutent(s) contains an organic functionality selected from alkyl, aryl, amine, amide, thiol, cyano, carboxyl, ester, olefinic, epoxy, silyl, nitro, and halogen.

Modified sol-gels includes at least partial cured (or gelled) preparations comprised of permeable metal oxide glass structures containing in addition to the sol-gel precursor materials, preferably one or more organic components that hydrolytically condense along with the sol-gel precursor such that the resultant sol-gel matrix imparts properties suitable for, by example, implantation. Suitable properties include low volume shrinkage over time, resistance to cracking and other physical defects, maintenance of protein function, and compatibility with the protein and/or reporter group, and compatibility with the animal or subject to which it may be implanted. Suitable organic materials include polyols such as glycerol, ethylene glycol, propylene glycol, polyethylene glycol, and the like, for example, as taught by Gill and Ballesteros Journal of the American Chemical Society 1998, 120(34), 8587-8598. Optimization of performance attributes of the protein-reporter pair and functional performance attributes of the encapsulating matrix may be achieved, for example, by way of combinatorial methods or other statistically based design methods known in the art.

A variety of polymers can offer barriers for the containment layer 2 (generally the second layer) and outer layer 3 (generally the third layer). While the third layer can provide a leaching barrier, it or a fourth layer may also provide biocompatibility. The additional outer layer or coating may be added to the multicoated or multilayer matrix to improve biocompatibility, for example, where the intended use is in vivo implantation. Some of the examples of various polymers that can be used for the optional fourth layer or coating are Nafion®, polyethylene glycol (PEG), poly (2-hydroxyethyl methacrylate) (pHEMA), and alginate.

In some instances, the multicoated or multilayer matrix of the invention may have only two layers or coatings—that is, a single composition may serve as both the containment layer 2 and the outer layer 3, or a containment layer may be unnecessary due to the properties of the core 1. When the core is of mixed composition, for example an inter-penetrating network, 2 to 3 layers of various materials may be sufficient.

One or more binding proteins for one or more analytes of interest may be incorporated into the core 1, optionally, along with a reporter group that provides a detectable signal upon analyte binding or interaction. For example, in one embodiment, mutated glucose/galactose binding proteins (GGBPs) comprises a detectable reporter group whose detectable characteristics alter upon a change in protein conformation that occurs on glucose binding. In a preferred embodiment, the reporter group is a luminescent label that results in a mutated GGBP with an affinity for glucose that exhibits a detectable shift in luminescence characteristics on glucose binding. The change in the detectable characteristics may be due to an alteration in the environment of the label bound to the mutated GGBP.

The term “binding protein” refers to a protein that interacts with a specific analyte in a manner capable of transducing or providing a detectable and/or reversible signal differentiable either from a signal in the absence of analyte, a signal in the presence of varying concentrations of analyte over time, or in a concentration-dependent manner, by means of the methods described herein. The transduction event includes continuous, programmed, and episodic means, including one-time or reusable applications. Reversible signal transduction may be instantaneous or time-dependent, provided a correlation with the presence or concentration of analyte is established. Binding proteins mutated in such a manner to effect transduction are preferred.

The binding protein may be any protein that is specific for an analyte of interest (including glucose, galactose, maltose, free fatty acid binding proteins and others) to which the analyte becomes reversibly bound or associated, and that can be induced to provide a signal, either directly or through a reporter group, when such binding occurs in the absence of a competitive ligand. Those of skill in the art know numerous binding proteins for sugars and other analytes of interest.

Particularly preferred for use in the invention are sugar binding proteins such as glucose, galactose and maltose binding proteins. The term “galactose/glucose binding protein” or “GGBP” or “maltose binding protein” or “MBP”, as used herein, refers to a type of protein naturally found in the periplasmic compartment of bacteria. These proteins are naturally involved in chemotaxis and transport of small molecules (e.g., sugars, amino acids, and small peptides) into the cytoplasm. For example, GGBP is a single chain protein consisting of two globular α/β domains that are connected by three strands to form a hinge. The binding site is located in the cleft between the two domains. When glucose enters the binding site, GGBP undergoes a conformational change, centered at the hinge, which brings the two domains together and entraps glucose in the binding site. X-ray crystallographic structures have been determined for the closed form of GGBP from E. coli (Vyas et al., Science 1988, 242, 1290-1295) and S. Typhimurium (Mowbray et al., Receptor 1990, 1, 41-54) and are available from the Protein Data Bank (http://www.rcsb.org/pdb/) as 2GBP and 3GBP, respectively. The wild type E. coli GGBP DNA and amino acid sequence can be found at www.ncbi.nlm.nih.gov/entrez/-accession number D90885 (genomic clone) and accession number 230520 (amino acid sequence). The preferred GGBP is from E. coli.

The binding protein may be any naturally occurring, engineered or mutated protein that specifically binds to the analyte of interest. “Mutated binding protein” (for example “mutated GGBP”), as used herein, refers to binding proteins from bacteria containing amino acid(s) that have been substituted for, deleted from, or added to the amino acid(s) present in naturally occurring protein. Preferably such substitutions, deletions or insertions involve fewer than five amino acid residues within the primary protein sequence. In addition to these changes, the mutated binding protein may also be combined with a fusion partner such as a polyhistidine sequence appended to the protein terminus for use during purification. Exemplary mutations of binding proteins include the addition or substitution of cysteine groups, non-naturally occurring amino acids (Turcatti et at., J Bio. Chem. 1996, 271 (33), 19991-19998) and replacement of substantially non-reactive amino acids with reactive amino acids to provide for the covalent attachment of electrochemical or photo-responsive reporter groups. By “reactive” amino acid is meant an amino acid that can be modified with a labeling agent analogous to the labeling of cysteine with a thiol reactive dye. Non-reactive amino acids include alanine, leucine, phenylalanine, and others, which possess side chains that cannot be readily modified once incorporated in a protein (see Greg T. Hermanson, Bioconjugate Techniques, Academic Press, 1996, San Diego, pp. 4-16 for classification of amino acid side chain reactivity). For example, a mutated glucose/galactose binding protein and reporter group as described in PCT patent applications PCT/US03/00200, PCT/US03/00201, and PCT/US03/00203, may be encapsulated or entrapped in an alginate core. The galactose/glucose binding proteins (GGBPS) that have been mutated to contain a cysteine residue, as described in the aforementioned PCT applications, are particularly preferred.

Exemplary mutations of the GGBP protein include: a cysteine substituted for a lysine at position 11(K11C); a cysteine substituted for aspartic acid at position 14 (D14C); a cysteine substituted for valine at position 19 (V19C); a cysteine substituted for asparagine at position 43 (N43C); a cysteine substituted for a glycine at position 74 (G74C); a cysteine substituted for a tyrosine at position 107 (Y107C); a cysteine substituted for threonine at position 110 (T110C); a cysteine substituted for serine at position 112 (S112C); a double mutant including a cysteine substituted for a serine at position 112 and serine substituted for an leucine at position 238 (S112C/L238S); a cysteine substituted for a lysine at position 113 (K113C); a cysteine substituted for a lysine at position 137 (K137C); a cysteine substituted for glutamic acid at position 149 (E149C); a double mutant including a cysteine substituted for an glutamic acid at position 149 and a serine substituted for leucine at position 238 (E149/L238S); a double mutant comprising a cysteine substituted for histidine at position 152 and a cysteine substituted for methionine at position 182 (H152C/M182C); a double mutant comprising a cysteine substituted for a glutamic acid at position 149 and a cysteine substituted for an alanine at position 213 (E149C/A213C); a double mutant including a serine substituted for an alanine at position 213 and a cysteine substituted for a histidine at position 152 (H152C/A213S); a cysteine substituted for an methionine at position 182 (M182C); a cysteine substituted for an alanine at position 213 (A213C); a double mutant including a cysteine substituted for an alanine at position 213 and a cysteine substituted for a leucine at position 238 (A213C/L238C), a cysteine substituted for an methionine at position 216 (M216C); a cysteine substituted for aspartic acid at position 236 (D236C); a cysteine substituted for an leucine at position 238 (L238C); a cysteine substituted for a aspartic acid at position 287 (D287C); a cysteine substituted for an arginine at position 292 (R292C); a cysteine substituted for a valine at position 296 (V296C); a triple mutant including a cysteine substituted for an glutamic acid at position 149, a serine substituted for a alanine at position 213 and a serine substituted for leucine at position 238 (E149C/A213S/L238S); a triple mutant including a cysteine substituted for an glutamic acid at position 149, an arginine substituted for an alanine at position 213 and a serine substituted for leucine at position 238 (E149C/A213R1L238S); a triple mutant including a cysteine substituted for an glutamic acid at position 149, a cysteine substituted for a alanine at position 213 and a cysteine substituted for leucine at position 238 (E149C/A213C/L238C); a quadruple mutant including a serine at position 1, a cysteine at position 149, an arginine at position 213 and a serine at position 238 (A1S/E149C/A213R/L238S); a quadruple mutant including a serine at position 1, a cysteine at position 149, a serine at position 213 and a serine at position 238 (A1S/E149C/A213S/L238S); and a quadruple mutant including a cysteine at position 149, a cysteine at position 182, a cysteine at position 213 and a serine at position 238 (E149C/M182C/A213C/L238S). Additional examples are listed in Table 2 hereinbelow. Amino acid residue numbers refer to the published sequence of E. coli having 309 residues, as detailed below, or the corresponding amino acid residue in any substantially homologous sequence from an alternative source (e.g., glucose/galactose binding proteins from Citrobacter freundii or Salmonella typhimurium, sequence accession numbers P23925 and P23905, respectively).

The term “coating” is used herein to be synonymous with the term “layer” in so much as to mean one or more layers or coatings homogeneously or heterogeneously dispersed, dispensed, physically or non-physically connected or in contact with, spatially disposed to each other, adjacent to, or integral with.

The term “energy source”, as used herein, refers to actinic radiation (i.e., electromagnetic radiation that can produce photochemical reactions, transitions, or processes in atoms or molecules). Such energy sources may produce or result in luminescence. Luminescence includes phosphorescence, fluorescence, and bioluminescence.

To “provide a detectable signal”, as used herein, refers to the ability to recognize a change in a property of a reporter group in a manner that enables the detection of, or corresponding concentration of an analyte of interest.

The reporter group to be included in the core of the matrix may be any group that will provide a detectable signal when the analyte of interest becomes associated with or bound to the binding protein. In one preferred embodiment, the reporter group is a fluorophore. As used herein, “fluorophore” refers to a molecule that absorbs energy and then emits light. Non-limiting examples of fluorophores useful as reporter groups in this invention include fluorescein, coumarins, rhodamines, tetramethylrhodamine-5-iodoacetamide (5-TMRIA), Quantum Red™, Texas Red™, Cy™-3, N-((2-iodoacetoxy)ethyl)-N-methyl)amino-7-nitrobenzoxadiazole (IANBD), 6-acryloyl-2-dimethylaminonaphthalene (acrylodan), pyrene, Lucifer Yellow, Cy™-5, Dapoxyl® (2-bromoacetamidoethyl)sulfonamide, (N-(4,4-difluoro-1,3,5,7-tetramethyl-4-bora-3a,4a-diaza-s-indacene-2-yl)iodoacetamide (BODIPY507/545 IA), N-(4,4-difluoro-5,7-diphenyl-4-bora-3a,4a-diaza-s-indacene-3-propionyl)-N-iodoacetylethylenediarnine (BODIPY® 530/550 IA), 5-((((2-iodoacetyl)amino)ethyl)amino)naphthalene-1-sulfonic acid (1,5-IAEDANS), carboxy-X-rhodamine, 5/6-iodoacetamide (XRIA 5,6) and fluorescence proteins such as green fluorescent protein. Preferably, IANBD is used.

Many detectable intrinsic properties of a fluorophore reporter group may be monitored to detect analyte binding. Some properties that may exhibit changes upon analyte binding include fluorescence lifetime, fluorescence intensity, fluorescence anisotropy or polarization, and spectral shifts of fluorescence emission. Changes in these fluorophore properties may be induced from changes in the fluorophore environment such as those resulting from changes in protein conformation. Environmentally-sensitive dyes such as IANBD are particularly useful in this respect. Other changes of fluorophore properties may result from interactions with the analyte itself or from interactions with a second reporter group, for example when FRET (fluorescence resonance energy transfer) is used to monitor changes in distance between two fluorophores.

Although the use of fluorescent labels is preferred, it is also contemplated that other reporter groups may be used. For example, electrochemical reporter groups can be used wherein an alteration in the environment of the reporter gives rise to a change in the redox state thereof. Such a change may be detected, for example, by use of an electrode. Other examples include non-natural amino acids, either as a linker to the reporter or as the reporter group itself.

The reporter group may be attached to the analyte binding protein by any conventional means known in the art. For example, the reporter group may be attached via amines or carboxyl residues on the protein. Covalent coupling via thiol groups on cysteine residues is particularly preferred. Any thiol-reactive group known in the art may be used for attaching reporter groups such as fluorophores to a cysteine of a binding protein. Iodoacetamide, bromoacetamide, or maleimide are well known thiol-reactive moieties that may be used for this purpose.

Fluorophores that operate at long excitation and emission wavelengths (for example, about 600 nm or greater excitation or emission wavelengths) are preferred when the molecular sensor is to be used in vivo, for example, incorporated into an implantable biosensor device (the skin being opaque below 600 nm). Presently, there are few environmentally sensitive probes available in this region of the spectrum and perhaps none with thiol-reactive functional groups. However, thiol-reactive derivatives of Cy-5 can be prepared, for example, as taught by Gruber et al., Bioconjugate Chem. 2000, 11, 161-166. Conjugates containing these fluorophores, for example, attached at various cysteine groups contained in mutated GGBPs, can be screened to identify which results in the largest change in fluorescence upon glucose binding.

The instant invention discloses methods of embedding, entrapping or encapsulating a binding protein specific for an analyte of interest. The method includes modifying binding proteins where directed labeling with a reporter group such as a fluorophore and/or covalent immobilization is achieved while retaining the analyte binding and signal producing properties of the binding protein. Covalent immobilization of binding proteins, such as GGBP, require a minimal impact on the conformational properties of the protein to enable its use as a biosensor. These conformational properties are necessary for GGBP binding protein to produce a detectable signal upon binding of ligand.

Methods for covalent attachment of binding proteins to biosensor matrices can be generally described as either: (a) directed covalent attachment methods where a covalent bond is formed between a specific amino acid residue of the binding protein and the matrix material, either directly, or through an appropriate linker molecule; or (b) random covalent attachment methods where one or more covalent bonds from a larger number of possible reactive sites on the binding protein are formed with the matrix either directly or through an appropriate linker.

Directed covalent attachment methods can use existing amino acids of a protein or they can use previously non-existing amino acids or derivatives introduced into the protein through protein engineering techniques such as site-directed mutagenesis. For example, one or more cysteines may be introduced within a protein sequence to provide a site with specific reactivity toward thiol-reactive reagents and linker groups with groups such as iodoacetamidyl, iodoacetoxyl, or maleimidyl functions. Such linker groups may be associated with or part of dye molecules or matrixes.

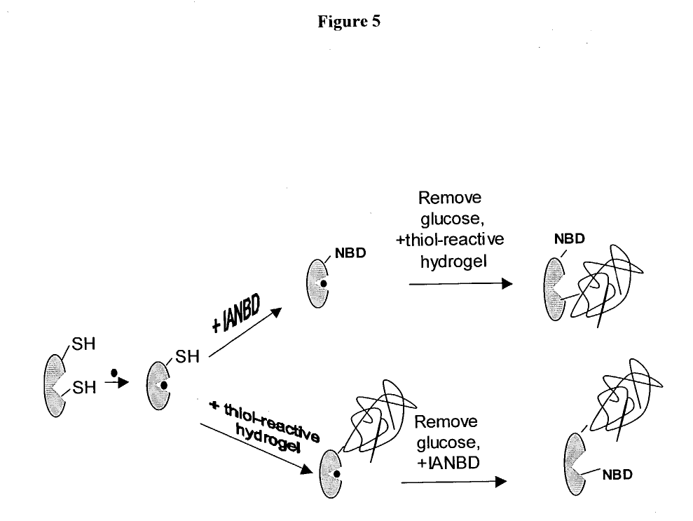

Chemical or reactive selectivity for one of two or more cysteines of a protein may be manipulated if one cysteine is significantly less accessible than the other(s). The instant method used herein, “ligand masking,” is defined as rendering at least one cysteine of the protein substantially less accessible from thiol-reactive reagents while the protein is in the presence of the ligand. The cysteines are sequentially modified, first in the presence, then in the absence of the appropriate ligand. Dye attachment and covalent immobilization can be done in either order as illustrated in FIG. 5.

A variation of the above mentioned approach is to introduce two cysteines within a protein so that at least one cysteine is substantially within a hydrophobic environment or region in the interior folds of a protein and at least another cysteine is at or near the more hydrophilic surface environment or region of the protein. By way of example, the protein is first modified using a hydrophilic dye or linker group that reacts preferentially with the cysteine in the hydrophilic surface region. A subsequent reaction with a hydrophobic dye or linker group forms a covalent attachment selectively to at least one unreacted cysteine within the hydrophobic region.

In another embodiment, an amine at the N-terminus of a protein can be selectively modified based on the difference between its pKa and the pKa of other amines such as ε-amine of lysine side chains. This may be achieved by controlling the pH of the protein environment during modification.

In another embodiment, by way of example, site-selective modification at the N-terminus of the binding protein may be achieved by selectively oxidizing the 1,2 amino alcohol group of a serine or a threonine in a first position with periodate to produce a glyoxal group at the N-terminus which can react with a hydrazide, hydrazine or aminooxy group containing linker group or matrix (Gaertner & Offord 1996; Alouani, et al, 1995; Geoghegan and Stroh, 1992). Particularly useful mutant binding proteins include, but are not limited to, A1S derivatives of GGBP.

In yet another embodiment, random covalent attachment methods of binding proteins to matrices or linker groups may involve one or more of a group of protein amino acids with similar reactivities. For example, protein lysines can be modified covalently through either alkylation or by acylation reactions with activated acyl groups such as N-hydroxy-succinimidyl esters. Glucose/galactose-binding protein (GGBP), for example, has multiple lysines on its surface that are available to react with such activated esters.

Suitable covalent attachment points for binding proteins onto matrices include, but are not limited to, the carboxylate groups of polysaccharides. Non-limiting examples include the carboxylate groups found on CMC (carboxymethylcellulose), glucuronic acid repeating units of hyaluronic acid, collagens, or the mannuronic and guluronic acid units of alginates. These exemplary carboxylate groups can be activated with 1-ethyl-3-(3-dimethylaminopropyl) carbodiimide hydrochloride (EDC) or EDC in combination with N-hydroxysuccinimide (NHS), or EDC in combination with N-hydroxysulfo-succinimide (sulfo-NHS). Another method for covalent attachment of binding proteins to polysaccharide matrixes is via periodoate oxidation of the polysaccharide vicinal diols. The resulting aldehyde groups may be reacted with amine groups on the binding protein and the resulting imines thus formed reduced with sodium cyanoborohydride to form stable secondary amine linkages between the protein and the polysaccharide matrix.

These examples are not intended to be limiting. Many additional methods for covalent attachment may be used for either random or specific attachment of binding proteins to a matrix or linker group. Other suitable general methods and strategies for attaching proteins to matrices have been described (Hoffman, 1996; Hermanson, 1996).

In one aspect of the present invention, the multicoated or multilayer matrix comprises a biosensor to be used for analyte sensing in vivo. In this aspect, the biosensor is encapsulated, entrapped, or embedded into the matrix that may then be used as an implantable device, either alone, or in combination with additional components. The matrix may be any desirable form or shape including one or more of bead, disk, cylinder, patch, nanoparticle, microsphere, porous polymer, open cell foam, providing it is permeable to analyte. The matrix additionally prevents leaching of the biosensor. Preferably, the matrix permits light from optical sources or any other interrogating light to or from the reporter group to pass through the biosensor. When used in an in vivo application, the biosensor will be exposed to a substantially physiological range of analyte, and determination or detection of a change in analyte concentration would be desired, whereas the determination or detection includes continuous, programmed, and episodic detection means. Thus, the envisaged in vivo biosensor of the present invention comprises at least one binding protein in an analyte-permeable entrapping or encapsulating matrix such that the binding protein provides a detectable and reversible signal when the binding protein is exposed to varying analyte concentrations, and the detectable and reversible signal can be correlated to the concentration of the analyte. The implantable matrix or device may be implanted into or below the skin of mammalian epidermal-dermal junction to interact with the interstitial fluid, tissue, or other biological fluids. Information from the implant to the patient or health care provider may be provided, for example, by telemetry, visual, audio, or, other means known in the art, as previously stated.

The encapsulating, embedding, or entrapping of the binding protein, optionally along with a reporting group, may be combined in a suitable solution that may also comprise a calibration dye.

The following examples illustrate certain preferred embodiments of the instant invention, but are not intended to be illustrative of all embodiments. Labeled mutated binding proteins with fluorophore reporter probes used herein in accordance with the procedure set forth by Cass et al., Anal. Chem. 1994, 66, 3840-3847, or as described.

Fluorescence emission spectra of a mutated, labeled protein was measured (unless otherwise stated) using an SLM Aminco fluorimeter (Ontario, Canada) with slit settings of 8 and 4 for excitation at 470 nm and settings of 5 and 5 on the MC250 emission monochromator to compare the ligand-binding performance of the entrapped fluorophore-labeled proteins in various matrices to the performance of the same proteins in solution. The initial fluorescence emission intensity is defined as I₀. The relative ratio of the emission intensity maxima in the presence of the protein's respective ligand (I) to the ligand's absence (I₀) is defined as I/I₀.

### EXAMPLE 1

### Multicoated or Multilayer Matrix

A: Without the Binding Protein in the Core

A multicoated or multilayer matrix of the invention was prepared in the following manner:

1. A core matrix was formed by mixing 1 part PBS buffer (pH 7.4) with 2 to 4 parts 3 wt % alginate (v/v) in a scintillation vial and vortexing at slow speed. 3 mL of the resulting alginate mixture was placed in a syringe and infused at a rate of 10 mL/hr into 200 ml of 1 M CaCl₂ on a Roto mix, thereby forming beads of about 0.4 to 1.5 mm in diameter. The beads were mixed in CaCl₂ solution on the Roto mix for 15-60 minutes.

2. A containment layer was formed by placing the beads from above in a solution of poly-L-lysine 0.01% w/v in water, approximately 10 mL, for 1 hour, then the poly-lysine coated beads were dried on an absorbent towel for 15 to 30 minutes.

3. The outer layer was formed by adding an additional 3 parts of buffer (v/v) to the GMSC solution, which was prepared as indicated below, then adding alginate/poly-L-lysine beads for about 12 minutes. The mixture was mixed slowly for 15 seconds, then the beads were poured onto an absorbent towel and dried for 15 to 30 minutes. The beads were stored in scintillation vials moistened with PBS (about 100 μL). A multicoated or multilayer matrix made by this method is illustrated in FIG. 1.

Glycerol modified silicate condensate (GMSC) sol-gel was prepared in advance using a modified procedure of Gill and Ballesteros (Journal of the American Chemical Society 1998, 120 (34), 8587-8598) with the following ratios of reagents: tetraethoxyorthosilicate (TEOS) or tetramethoxyorthosilicate (TMOS): 1; H₂O: 1, methanol:4, glycerol:1. TEOS or TMOS in methanol was added to a flask and cooled to 0° C. over ice. 4.1 mL of 0.6 M HCl was then added drop-wise to the solution. After 20 minutes of stirring, glycerol was added dropwise. The reaction was warmed slowly over 1-2 hours to 20-25° C. Following this, the reaction vessel was heated further and maintained at a temperature range of 60-70° C. under nitrogen for between 36 and 42 hours (optimally 40 hours). Following the reaction at 60-70° C., the solution volume was reduced by rotary evaporation until it was viscous and transparent, at which point methanol was added to the solution in a 4:1 ratio by weight. This GMSC solution was stable and provides consistent results for several months when stored at freezer temperature. When the GMSC solution was to be used, methanol was removed by rotary evaporation, and distilled water was added in a 1:1 ratio by weight to the GMSC reagent.

B: With the Entrapped Binding Protein

A multicoated or multilayer matrix of the invention was prepared in the following manner:

1. A core matrix was formed by mixing 1 part dye-labeled binding protein (15 uM in PBS buffer, pH 7.4, prepared as described in PCT/US03/00203) with 2 to 4 parts 3 wt % alginate (v/v) in a scintillation vial and vortexing at slow speed. 3 mL of the resulting protein-alginate mixture was placed in a syringe and infused at a rate of 10 mL/hr into 200 ml of 1 M CaCl₂ on a Roto mix, thereby forming beads of about 0.4 to 1.5 mm in diameter. The beads were mixed in CaCl₂ solution on the Roto mix for 15-60 minutes. (The nature of the divalent cation used gives slightly different properties to the resultant core. For example, calcium cured beads produced greater change in fluorescence (I/I₀=2.5+/−0.17) than barium cured beads (I/I₀=1.9+/−0.29)).

2. A containment layer was formed by placing the beads from above in a solution of poly-L-lysine 0.01% w/v in water, approximately 10 mL, for 1 hour, then the poly-lysine coated beads were dried on an absorbent towel for 15 to 30 minutes.

3. The outer layer was formed by adding an additional 3 parts of buffer (v/v) to the GMSC solution (as prepared in the preceding example), then adding alginate/poly-L-lysine beads for about 12 minutes. The mixture was mixed slowly for 15 seconds, then the beads were poured onto an absorbent towel and dried for 15 to 30 minutes. The beads were stored in scintillation vials moistened with PBS (about 100 μL).

### EXAMPLE 2

### Effect of the Outer Layer on Protein Leaching from the Multicoated or Multilayer Matrix

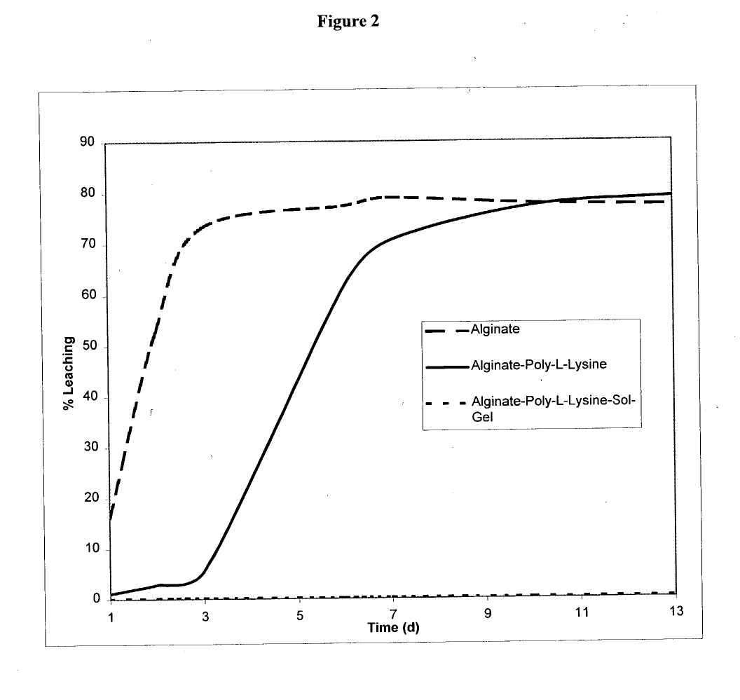

Multicoated or multilayer matrices were prepared as in Example 1B. One gram of beads were put in a scintillation vial with 3 mL of PBS buffer. The concentration of dye-labeled protein in one gram of beads was calculated to be 200 uM based on the dye absorbance. The vials with the beads were shaken on a vortex. Readings were taken at various time intervals. At each time point an aliquot of the buffer was removed and a reading taken in the fluorimeter. The lower detection limit was established at 0.05 uM. The results shown in FIG. 2 demonstrate that a thin layer of sol-gel is sufficient to provide undetectable leaching levels of the protein.

### EXAMPLE 3

### Effect of Various Polymer Compositions on Matrix Performance

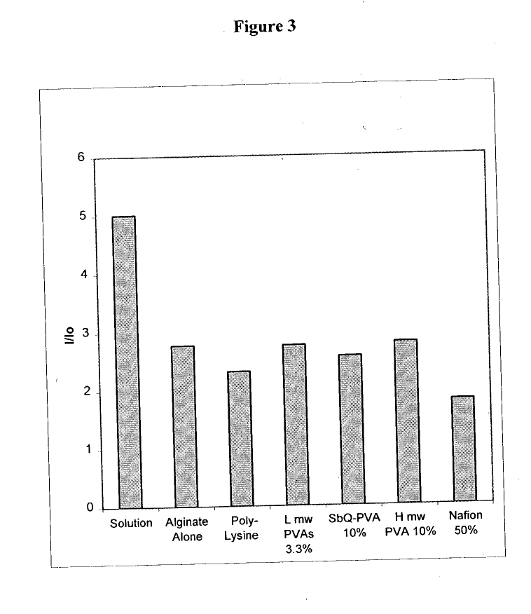

Multicoated or multilayer matrices were prepared as in Example 1 using a variety of polymers to form the containment layer 2. The resultant products from Example 1 were exposed to aqueous solutions of the following: (a) low molecular weight PVA (<5 k mw) (1 to 10% w/v); (b) high molecular weight PVA (>20 k mw) (1 to 10% w/v); (c) Nafion® (0.1 to 5.0% v/v); and (d) SbQ-PVA (1 to 50% v/v). The I/I₀ ratio was measured as described above. FIG. 3 shows the effects of different polymers used as a second layer on a core matrix comprised of NBD-GGBP entrapped in alginate. The I/Io response for glucose is shown for NBD-GGBP in solution, entrapped in alginate, and with the second layers. The results, shown in FIG. 3, demonstrate a variety of polymers are effective as the containment layer.

### EXAMPLE 4

### Multicoated or Multilayer Matrix with a Mixed Core

An exemplary mixed core multicoated or multilayer matrix according to the invention may be prepared by mixing 2 parts binding protein solution (15 uM in buffer) to 2 to 4 parts 3 wt % alginate and 1 to 2 parts of a light curable polymer (SbQ-PVA, 13.3 wt % in water) plus an additional step where the mixed matrix is exposed to a light source emitting between 500 and 600 nm and processing as in steps 2-3 of Example 1A or 1B. The mixed matrix can be formed with various light or UV curable materials such as SbQ-PVA and HEMA. This mixing of photocurable polymers provides tailoring of the structure of the core. For example, the crosslink density of the core can be varied by adjusting the ratio of the components used, light intensity, and exposure time to alter the core's rigidity.

### EXAMPLE 5

### Inter-Penetrating Network Core Multicoated or Multilayer Matrix

An inter-penetrating network core matrix may be prepared by mixing 2 parts of a polymer, to 2 to 4 parts 3% alginate, and processing as in steps 2-3 of Example 1, if necessary, depending on the number of layers to be added. This method makes it possible for the second polymer to physically hold the structure of the alginate. It will minimize the swelling of the hydrogel and will provide for elimination or resistance to leaching.

### EXAMPLE 6

### Use of Multicoated or Multilayer Matrix to Measure Glucose Concentration in Blood and Serum

Multicoated or multilayer matrix beads were prepared following the procedure described in Example 1, using one part binding protein solution to 4 parts 3 wt % alginate for the core, 0.01 wt % poly-L-lysine solution to form the second layer, and one part sol-gel to 2 parts (w/v) PBS (pH 7.4) to form the outer layer. The binding protein solution consisted of approximately 20 ul of GGBP (about 13 uM) in PBS or Tris buffer. This binding protein is specific for glucose, and the reporter group fluoresces in response to glucose binding.

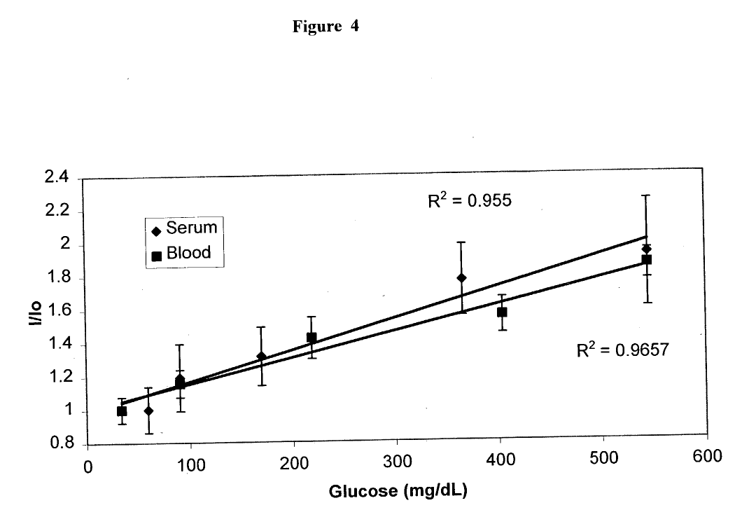

After drying the beads, about 6 beads were glued with Dymax 128M to the bottom of each well of a UV transparent black 96 well plate and allowed to cure under ambient conditions. A Cyto Fluor series 400 fluorescent plate reader was used for the assay. The excitation filter was 485/20 nm and the emission filter was 530/20 nm. To the wells were added 100 μl of rabbit blood or serum, and fluorescence was read using the plate reader. The original samples contained a base concentration of glucose and other glucose levels were obtained by adding concentrated glucose solutions in PBS. The concentrations of glucose were also determined with a YSI analyzer for comparison. The results, shown in FIG. 4, demonstrate that the multicoated beads are stable and maintain the binding protein's response proportional to the concentration of glucose. This response is robust even in the presence of complex biofluids such as blood and serum.

### EXAMPLE 7

### Selective Attachment of Binding Protein Through an N-Terminal Serine or Threonine to Matrix

A solution of the A1S/E149C/A213R/L238C-His₆ mutant of GGBP is prepared in either PBS (pH 7.2) or 0.1 M NH4HCO₃ (pH 8.3) with a 50-fold excess of methionine (as a scavenger). A 10-fold molar excess of sodium periodate in water is added and the solution is incubated in the dark for 10 min. The reaction is quenched by addition of ethylene glycol (20,000-fold molar excess) or sodium sulfite (25-fold molar excess), and the buffer is exchanged with 0.1 M sodium acetate (pH 4.6) either by dialysis or by passing through a NAP-5 column. The protein solution is added to a polymer or matrix (10-fold molar excess) containing a hydrazide, hydrazine or aminooxy group which reacts with the glyoxal of the protein. The reaction is allowed to proceed for 1-24 hours and the product is purified by washing the reaction mixture five times with sodium acetate buffer. The resulting conjugate can be reduced with NaBH₃CN to enhance stability of the linker.

### EXAMPLE 8

### Directed Modification of Multiple Cysteines of A Binding Protein by Ligand Masking

Various cysteine-containing GGBP mutants were labeled with IANBD in the presence and absence of glucose. For IANBD dye labeling, between 4 and 10 nmoles GGBP protein was prepared in PBS buffer in a microcentrifuge tube (0.5 to 1.5 mg/mL) and 2.5 molar equivalents of DTT per cysteine on the protein was added. After mixing for 20-30 minutes, the solution was divided into two further tubes to which glucose (final concentration 87 mM) or an equivalent volume of buffer was added and the solutions were mixed for another 15 to 30 minutes (ligand masking step). The final concentration of glucose was chosen to saturate most of the glucose binding sites in the GGBP mutant. For the dye labeling, IANBD was added as a 0.5 mg/mL solution, in DMSO (ten equivalents of IANBD per cysteine) to each tube and all solutions were mixed for an hour in the dark. The dye-labeled protein was then separated from free dye by elution from a NAP-5 size exclusion column eluting with PBS buffer. The dye:protein ratio was determined by comparison of absorbance spectra of the eluted protein fractions. Results of the ligand masking experiment labeling seveal GGBP mutant proteins with glucose (+) and without glucose (−) are shown in Table 1.

TABLE 1
Ligand masking of GGBP proteins

| | | Dye: Protein Ratio | |
|---|---|---|---|
| | Final concentration | − | + |
| GGBP mutant | glucose (mM) | glucose | glucose |
| E149C | 16 | 0.3 | 0.3 |
| E149C/A213R/L238S | 107 | 1 | 1 |
| A213C/L238C | 107 | 1.8 | 1.2 |
| A213C | 99 | 1.3 | 0.4 |
| L238C | 99 | 0.5 | 0.2 |

As shown in Table 1, the difference in labeling efficiency in the presence vs. absence of glucose is greatest for the GGBP mutants having the A213C and L238C mutations. This indicates these cysteines can be rendered less accessible for reaction by the ligand masking strategy. By comparison, the accessibility of the E149C site to reaction with dye does not appear to be significantly changed by the presence of the ligand glucose As the dye:protein ratio is the same (0.3) with or without glucose present.

Additional GGBP mutants with at least two cysteines were successfully labeled with NBD. . Based on the results from Table 1 it is likely most dye-labeleing in the presence of glucose occurred at the E149C location on mutants E149C/A213C/L238C and E149C/A213C. The results, summarized in Table 2, demonstrate directed labeling of the mutant binding protein by the instant method.

TABLE 2
Ligand masking of GGBP with more than one cysteine mutation.

| | | Dye: Protein Ratio | |
|---|---|---|---|
| | Final concentration | − | + |
| GGBP mutant | glucose (mM) | glucose | glucose |
| E149C/A213C/L238C | 87 | 2.8 | 2 |
| E149C/A213C | 87 | 2.1 | 1.3 |

Selective attachment of protein through remaining free cysteine to CMC is described here. Residual glucose is removed from the masked-labeled protein prepared by Example 8 by exhaustive dialysis. To insure the remaining cysteine is free to react, prior to the immobilization, the dialyzed protein is treated with immobilized tris[2-carboxyethylphosphine] hydrochloride (TCEP) reagent (on beads, Pierce Chemical) and the reduced protein is separated using a spin column.

The thiol-reactive CMC hydrogel is prepared as follows. Equal volumes of 100 mM NHS and 400 mM EDC are combined; the EDC/NHS mix is combined with carboxymethyl cellulose (CMC) for 15 minutes at room temperature. (Final concentration is CMC, 13 mg/mL; 26 mM NHS; 204 mM EDC). Unreacted EDC/NHS is removed with a NAP-5 column, further diluting the activated CMC to 6.5 mg/mL. Activated CMC is combined with 2-(2-pyridinyldithio)-ethaneamine (PDEA) for 15 minutes at room temperature (300 uL activated CMC, 6.5 mg/mL +200 uL PDEA (18 mg/mL in 0.1M sodium borate, pH 8.5)). Unreacted PDEA is removed using a NAP-5 column. This further dilutes the PDEA-activated CMC to 2 mg/mL.

PDEA-activated CMC is added to between 0.05 and 1.5 mg/mL solution of labeled protein and mixed 2.5 hours, at room temperature. Cysteine (100 uL of 50 mM cysteine in 0.1M sodium formate, pH 4.3, 1M NaCl), is added to each tube, and mixed for 15 minutes at room temperature to quench the reaction. Unreacted cysteine is removed by NAP-5 column to give the CMC-protein conjugate.

### EXAMPLE 9

### Random Covalent Attachment of Binding Protein to Carboxymethyl Cellulose

The following hydrogel material was used for the covalent coupling of binding protein: sodium carboxymethyl cellulose (NaCMC) containing 0.7 moles COOH per mole cellulose (mw about 90,000 Sigma-Aldrich, St. Louis, Mo.).

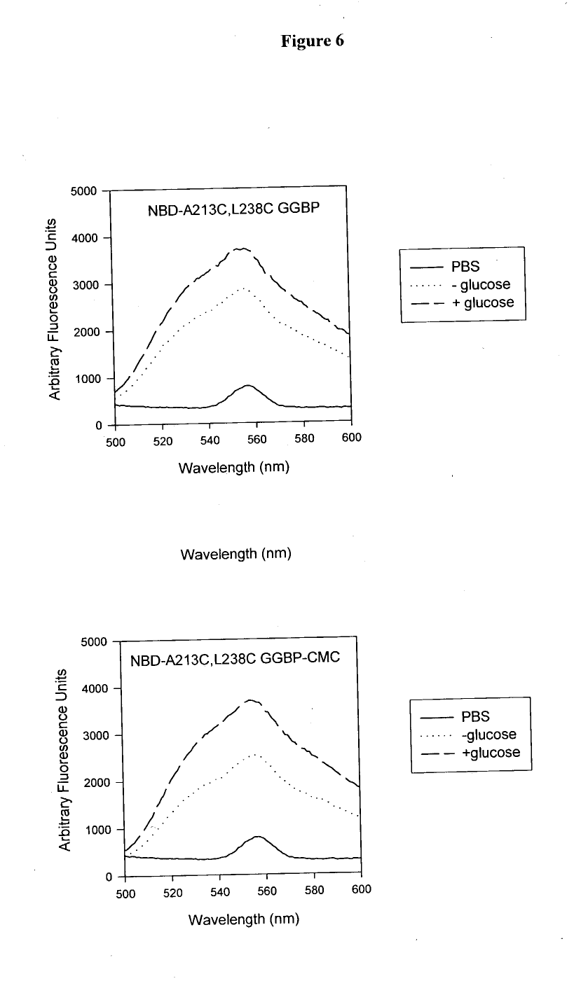

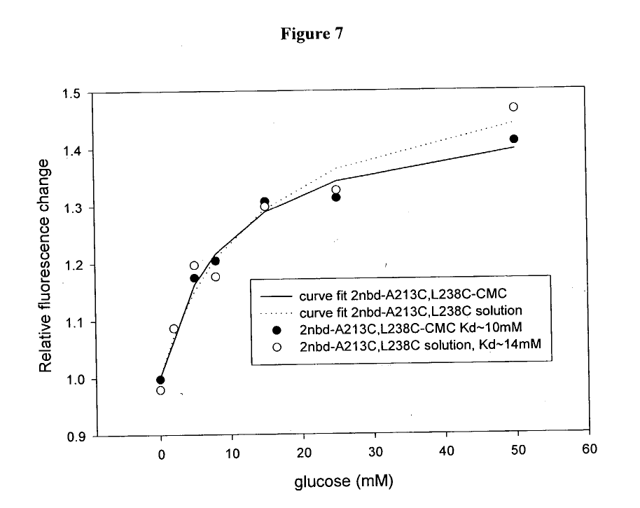

Covalent attachment was performed by activating carboxymethyl groups on the hydrogel polymer (CMC) with a mixture of N-hydroxysuccinimide (NHS) and N-ethyl-N′-(dimethylaminopropyl) carbodiimide (EDC). This forms an intermediate that is reactive to amines. NHS (100 mM) and EDC (400 mM) in water were mixed in a 1:1 vol:vol ratio. An equal volume of hydrogel polymer (25 mg/mL CMC in H₂O) and the EDC/NHS mixture were combined, and incubated at room temperature for 15 to 30 minutes. The unreacted EDC and NHS were removed from the hydrogel polymer by eluting the polymer from a NAP-5 column. Varying amounts of activated hydrogel polymer were combined with the fluorescently-labeled GGBP (in this example, the NBD-A213C/L238C-GGBP-His6 mutant). The activated hydrogel polymer and protein were gently mixed, at room temperature, for 1 to 2 hours. The reaction was quenched by the addition of 1 M ethanolamine-HCl, pH 8.5, followed by removal of ethanolamine by elution from a NAP-5 column. Microfuge filters with a molecular weight cutoff of 100 kDa were used to concentrate the GGBP-linked hydrogel polymers. An absorbance scan of solution not retained by the 100 kDa filter indicated no unattached protein remained. The concentrated NBD-GGBP-CMC in solution was examined using protein, gel electrophoresis. Agarose gels (1% and 1.5% in 1×TBE (0.1M Tris, 0.09M boric acid, 0.001M EDTA, pH 8.4), running buffer of 1×TBE with 0.1% SDS) demonstrated that most of the protein was attached to the hydrogel matrix as indicated by the NBD-GGBP-CMC conjugate migrating as a smear with an apparent high molecular weight (between the 97 kDa and 390 kDa markers). A control lane with unconjugated GGBP migrated with an apparent molecular weight below the lowest mw marker (97 kDa). Activity of the NBD-GGBP-CMC was determined by fluorescence intensity measurement of the NBD-GGBP-CMC in the presence of high concentration glucose (20 mM) and in the absence of glucose. Thus, 100 nM preparations of the NBD-GGBP-CMC, as well as unattached NBD-GGBP (control) were measured before and after the addition of glucose (FIG. 6). The NBD-GGBP and NBD-GGBP-CMC materials were also titrated against varying amounts of glucose. Using 110 nM NBD-GGBP or NBD-GGBP-CMC in PBS buffer, aliquots of varying glucose concentration were added to maintain a final protein concentration of 100 nM. The resultant equilibrium dissociation constants (FIG. 7) for the hydrogel polymer-linked protein and solution phase protein versus glucose were similar (10 mM and 14 mM respectively). The data demonstrates the NBD-GGBP-CMC complex retained the requisite ligand-induced conformational property and provided a strong signal in the presence of glucose compared to the absence of glucose clearly demonstrating the utility of the complex as a viable biosensor.

### EXAMPLE 10

### Covalent Random Attachment of Glucose Binding Protein to Alginate Core Matrix

Crosslinked alginate-based scaffolds were prepared by covalently crosslinking Pronova™ UP LVG (low viscosity, high guluronic to mannuronic ratio) alginate (FMC Biopolymers) through the carboxyls with adipic acid dihydrazide (AAD) via carbodiimide chemistry. For 2% alginate hydrogels and scaffolds, 1 gram of alginate was dissolved in 50 mL 0.1 M MES buffer (pH 6.0), to which 110 mg of AAD and 79 mg of hydroxybenzotriazole (HOBt) were added. The solution was stored at 4° C. until used. To the alginate solution 145 mg of EDC (per 10 mL solution) was added using a dual-syringe mixing technique, and the mixed solution was cast between parallel glass plates with 2 mm spacers and allowed to gel for 3 hrs. For the 3% alginate hydrogels and scaffolds, the quantities of AAD, HOBt and alginate were multiplied by 1.5×. A 5 mm biopsy punch was used to cut out 2×5 mm disks, that were washed extensively in water to remove salts and any reactants (36-48 hr, agitated with 5-6 water changes). The hydrogel disks were placed on a polypropylene surface, frozen overnight at −20° C. and lyophilized to create the open pore structure of the scaffold. Dry weight of each disk was approximately 1 mg for 2% scaffolds and contained a theoretical minimum of 3.8 umol of COOH available for modification by EDC/NHS.

Covalent attachment of binding protein to covalently crosslinked alginate hydrogels and lyophilized scaffolds were performed as follows:

1. Carboxyl groups on the alginate scaffold prepared as above were activated with a mixture of N-hydroxysuccinimide (NHS) and N-ethyl-N′-(dimethylaminopropyl) carbodiimide (EDC) followed by attachment of labeled GGBP protein mutants [(NBD-E149C/A213R/L238S- or NBD-E149C/A213R-GGBP)] as follows:

A. Method 1: A solution of labeled GGBP in PBS buffer [NBD-E149C/A213R/L238S GGBP] (53 uM, 100 uL) was incubated with the lyophilized scaffold for 30 min, followed by removal of about 60 uL of the protein solution. The scaffolds were infused with 40 uL of EDC/NHS (200 mM/50 mM) or PBS (no EDC negative control) and incubated for 60 min.

B. Method 2: A solution of EDC/NHS (200 mM/50 mM, 100 uL) was infused into the lyophilized scaffold, and incubated for 15 minutes followed by removal of about 60 uL of the solution followed by the infusion of 40 uL of labeled GGBP (53 uM) and incubated for 60 minutes.

After incubation, the scaffolds of Method 1 or 2 were washed (1× at 1 h, 1× at 64 h) in approximately 10 mL PBS. The fluorescence of the scaffolds in the presence and absence of glucose was measured with a Cytofluor instrument (as described in Example 6), showing an increase in fluorescence with glucose. The following table demonstrates the GGBP protein covalently attached to the alginate scaffolds provides a fluorescence response to varying glucose concentration.

TABLE 3
Fluorescence response of [NBD-E149C/A213R/L238S GGBP] alginate
scaffolds to glucose.

Fluorescence Response
(arbitrary units)

| Scaffold | 0 mM Glucose | 30 mM Glucose |
|---|---|---|
| Blank | 41 | 52 |
| Blank | 42 | 42 |
| Method 2 | 3176 | 6300 |
| Method 2 | 2660 | 4500 |
| Method 1 | 5479 | 10500 |
| Method 1 | 4462 | 10200 |
| No EDC | 102 | 119 |
| No EDC | 95 | 109 |

No EDC means that the protein was added, but no covalent chemistry was performed. Samples were stored in PBS for 72 hours before use. Results are shown for duplicate experiments.

### EXPERIMENT 11

### Hydrogel Experiments

Labeled GGBP (0.85 mg/mL in PBS, 50 uL each hydrogel disk) was incubated with the 2% or 3% covalently cross-linked alginate hydrogels for 90 minutes. The hydrogel disks were infused with solutions of EDC/NHS (200 mM/50 mM, 50 uL) and incubated for 2 hours. Other hydrogels were infused with 50 uL water as a negative control. The reaction was quenched with 10 uL of 1M ethanolamine, pH 8.5 (Biacore AB) for 15 minutes. After incubation and quenching, the scaffolds were washed in approximately 1 mL PBS each for 1 hour. The fluorescence of the scaffolds in the presence and absence of saturating amounts of glucose was measured, and the data showed fluorescence activity with respect to glucose concentration (see Table 4).

TABLE 4
Fluorescence response of [NBD-E149C/A213R GGBP] alginate
hydrogels to glucose.

| | | Fluorescence Signal, | | |
|---|---|---|---|---|
| | | background subtracted | | |
| Alginate | Attachment chemistry | No glucose | 48 mM glucose | I/Io |
| 2% | EDC/NHS, (covalent) | 293 | 1122 | 3.8 |
| 2% | None | 63 | 236 | 3.8 |
| 3% | EDC/NHS, (covalent) | 359 | 1270 | 3.5 |
| 3% | None | 66 | 318 | 4.8 |

Thus, while incubation of the protein and the hydrogel without EDC/NHS seems to cause some physical entrapment of the protein, covalent attachment with EDC/NHS significantly increases the amount of protein that is retained.

### EXAMPLE 12

### Covalent Random Attachment of Binding Protein to Hydrogels that Are Covalently Linked to Amine-Coated Surfaces

All incubations were at room temperature. Poly-L-lysine (PLL, 19.6 mg/mL in H₂O, 50 uL) was incubated (2 hrs) in 96-well plates (BD Falcon, tissue culture treated), to create an amine-coated surface. The PLL-coated wells were washed with 200 uL H₂O, three times, to remove unattached PLL. The hydrogel (alginate) was combined with an EDC/NHS mixture as follows, to create a hydrogel that is reactive with amines. The EDC/NHS was prepared by combining 31 mg EDC, 21.5 uL 0.1M MES (pH 6.5), and 7 uL of NHS (0.6 g/mL in H₂O). Alginate (0.23 mL of 3% alginate in H₂O, Pronova™ UP LVG (low viscosity, high guluronic to mannuronic ratio, FMC Biopolymers) was combined with 35 uL of 1M MES (pH 6.75) and 12 uL of the EDC/NHS mixture above. An aliquot (40 uL) of the EDC-activated alginate was added to several wells and incubated for 15 minutes. At this point, the order of reagents was varied, for different samples, as described in Table 5 below. These reagents included the covalent crosslinker adipic acid dihydrazide (AAD, 10 uL, 200 mM in H₂O), the glucose binding protein NBD-GGBP (E149C/A213R/L238S mutant, 10 uL, 46 uM in PBS), and an ionic crosslinker, CaCl₂ (3.5 uL, 100 mM in H₂O).

After these reactions, the wells were washed with 150 uL PBS (10 minute incubation). Ethanolamine (10 uL of a 1M aqueous solution, pH 8.5) was added to each well to quench the reaction (20 minute incubation). Wells were washed three times with 150 to 200 uL PBS or PBS with 1 mM CaCl₂ (if CaCl₂ was used, as indicated in Table 5). PBS or PBS with 1 mM CaCl₂ (50 uL) was added to the wells for the fluorescence reading with the Cytofluor instrument. After this reading, 10 uL of 1 M glucose was added to the wells (30 minute incubation), and then another fluorescence measurement was taken. Results given in Table 5 show the response of the immobilized binding protein to glucose as I/Iₒ.

TABLE 5
Immobilization of NBD-GGBP on poly-L-lysine/alginate

| | Addition 1 | Addition 2 | Addition 3 | Fluorescence Intensity | | |
|---|---|---|---|---|---|---|
| | (15 min | (15 min. | (75 | (Background subtracted) | | |
| Experiment | incubation) | incubation) | min incubation) | −glucose | +glucose | I/Iₒ |
| #1 | AAD | NBD-GGBP | CaCl₂ | 115 | 454 | 3.9 |
| #2 | AAD | NBD-GGBP | — | 136 | 587 | 4.3 |
| #3 | NBD-GGBP | AAD | CaCl₂ | 178 | 454 | 2.6 |
| #4 | NBD-GGBP | AAD | — | 160 | 424 | 2.7 |
| #5 | CaCl₂ | AAD | NBD-GGBP | 87 | 421 | 4.8 |
| #6 | CaCl₂ | NBD-GGBP | AAD | 144 | 443 | 3.1 |

The results of the above experiment demonstrate random covalent attachinent of a protein to a matrix that is covalently attached to an amine-containing surface. This data indicates that binding protein, for example NBD-GGBP, can be covalently attached through its surface lysines, to a hydrogel polymer that is covalently attached to an amine-containing surface. The data showed fluorescence activity with respect to glucose concentration. The complex of NBD-GGBP/hydrogel polymer/amine surface was cross-linked, covalently, ionically, and/or both, to add structural stability to the matrix, increase concentration of protein, and/or reduce or prevent leaching of the protein. The cross-linking of layers and the covalent attachment of protein may be carried out sequentially or can be a simultaneous reaction; the order of attachment, timing of such reactions, and combination of reagents can affect the amount of protein immobilized, and the activity of the protein.

### EXAMPLE 13

### Random Attachment to Polymer Matrix Through Acrylate Groups Covalently Attached to a Protein

Examples of bifunctional linker groups include but are not limited to N-acryloyl succinimides, alpha-acryloyl omega-succinimidyl esters of polyethylene glycol propionic acid, and acryloyl-amino-hexanoic acid succinimidyl esters. Specific examples include ACRL-PEG-NHS (3400 MW alpha-acryloyl, omega-succinimidyl ester of polyethylene glycol propionic acid, (product number 012Z0F02, Shearwater Corp., Huntsville Ala.) and acryloyl-X SE (6-((acryloyl)amino)hexanoic acid, succinimidyl ester (product number A-20770, Molecular Probes, Eugene, Oreg.), and N-acryloyl succinimide (NAS, product A8060, Sigma-Aldrich, St. Louis, Mo.).

Any of the aforementioned reagents or equivalent heterobifunctional reagents may be used and are within the scope of the instant invention. These reagents react with lysine amines on the surface of the protein via the succinimidyl (NHS) ester and can then be co-polymerized with appropriate copolymers which react through the acryloyl functionality. These two general reactions may be done in either order for random covalent immobilization of a binding protein. Sequential and simultaneous reactions are contemplated.

Reaction of protein with succinimidyl group of NAS prior to co-polymerization. The dye-labeled binding protein (1-10 mg/mL) is dissolved in 0.2-0.3 M carbonate buffer with 1 M NaCl (pH 9.3). NAS (5 mg/mL) is added and the solution is mixed for 60 minutes. The reaction is quenched with ethanolamine or with tris buffer and the acryl-modified protein is recovered by elution from a NAP-5 column. Characterization of the number of acrylate groups per protein may be performed by isoelectric focusing gels as described by Shoemaker, et al. (1987). The modified protein is then added as a 1-10 mg/mL solution to a polymerization reaction with a suitable monomer such as hydroxyethyl methacrylate (HEMA), an initiator such as N,N-dimethyl para-toluidine or azo-bisisobutyronitrile (AIBN), and 1-5% of a crosslinking reagent such as trimetilol propane triacrylate or tetraethyleneglycol diacrylate (TEGDA). Polymerization is carried out by exposure of the mixture to light or heat, depending on the initiator, under anaerobic conditions for a sufficient time to produce a hydrogel polymer with randomly attached binding protein.

Co-polymerization of NAS in hydrogel to provide a protein-reactive matrix. A similar copolymerization step is performed as in the last step above but using NAS instead of the acryl-modified binding protein. The NAS is typically added as approximately 1-5% of the total monomer ratio. This produces a hydrogel polymer with reactive NHS esters. This material is placed in equilibrium with a solution of the NBD-labeled binding protein (1-10 mg/mL) for up to 24 hours, followed by washing with buffer. This produces a random covalent attachment of the protein to the copolymer by reaction of the NHS groups incorporated within the copolymer with protein that diffuses into the polymer matrix.

References:

Gaertner, H. F. and Offord, R. E. “Site-specific attachment of functionalized poly(ethylene glycol) to the amino terminus of proteins” Bioconj. Chem. 1996, 7, 38-44.

Alouani, S., Gaertner, H. F., Mermod, J.-J., Power, C. A., Bacon, K. B., Wells, T. N. C., and Proudfoot, A. E. T. “A fluorescent interleukin-8 receptor probe produced by targeted labeling at the amino terminus.” Eur. J. Biochem. 1995, 227, 328-334.

Geoghegan, K. F. and Stroh, J. G. “Site-directed conjugation of nonpeptide groups to peptides and proteins via periodate oxidation of a 2-amino alcohol. Application to modifications at N-terminal serine.” Bioconj. Chem. 1992, 3, 136-146.

A. S. Hoffman, “Biologically Functional Materials” chapt. 2.11, pp. 124-130, in “Biomaterials Science: An Introduction to Materials in Medicine”, B. D. Ratner, A. S. Hoffman, F. J. Shoen, J. E. Lemons, editors, Academic Press, San Diego, Calif., 1996.

G. T. Hermanson, “Bioconjugate Techniques”, pp. 605-630, Academic Press, San Diego, 1996.

Shoemaker, S. G, Hoffman, A. S., and Priest, J. H. “Synthesis and Properties of Vinyl Monomer/Enzyme Conjugates”, Appl. Biochem. Biotechnol. 1987, 15:11-24.

What is claimed is:

1. A multicoated or multilayer matrix capable of entrapping, embedding or encapsulating a binding protein comprising:
a core capable of physically or chemically entrapping a binding protein, and
one or more of: a containment layer that ensures the integrity of said core,
an outer layer selectively permeable to an analyte of interest, or
a layer that is biocompatible.

2. The matrix of claim 1, comprising a
core capable of physically or chemically entrapping a binding protein,
a containment layer that ensures the integrity of the core, and
an outer layer selectively permeable to an analyte of interest.

3. The matrix of claim 2, comprising the core and a layer that is biocompatible.

4. The matrix of claim 1, wherein said core comprises a cross-linkable polymer.

5. The matrix of claim 4 wherein said cross-linkable polymer is alginate or hyaluronate.

6. The matrix of claim 1, wherein said containment layer comprises a compound selected from the group consisting of poly-L-lysine, poly-D-lysine, low and high molecular weight polyvinyl alcohols, SbQ-PVA, and Nafion®.

7. The matrix of claim 1 wherein said outer layer comprises a compound selected from the group consisting of SbQ-PVA, HEMA, PVA, alginate and inorganic or organically modified inorganic sol-gels.

8. The matrix of claim 2 wherein said fourth layer comprises a compound selected from the group consisting of Nafion®, polyethylene glycol (PEG), poly (2-hydroxyethyl methacrylate) (pHEMA), and alginate

9. The matrix of claim 1, wherein said core comprises a binding protein.

10. The matrix of claim 9, wherein said binding protein is physically entrapped in said core.

11. The matrix of claim 9, wherein said binding protein is covalently bound to said core.

12. The matrix of claim 9 wherein said binding protein is a sugar binding protein.

13. The matrix of claim 12, wherein said sugar binding protein is a mutated glucose/galactose or maltose binding protein.

14. The matrix of claim 9, wherein said binding protein additionally comprises a reporter group.

15. The matrix of claim 14, wherein said reporter group is a luminescent or fluorescent group.

16. A method of making a multicoated or multilayer matrix comprising the steps of:
(a) forming a core;
(b) contacting the core with a first coating solution for a sufficient time to coat or layer the core;
(c) isolating and drying the first coating on the core to form the containment layer or coating;
(d) contacting the containment layer or coating with a second coating solution to form a multi-coated three dimensional structure; and
(e) isolating and drying the second coating on the multi-coated three dimensional structure to obtain a multicoated or multilayered matrix.

17. A method for measuring a concentration of an analyte in a solution comprising the steps of:
(a) contacting the matrix of claim 14 with a solution;
(b) exposing the matrix to an energy source capable of producing a detectable signal therefrom;
(c) measuring the detectable signal; and
(d) correlating the measure of the detectable signal with the concentration of analyste in the solution.

18. The method of claim 17, wherein said analyte is a sugar.

19. The method of claim 16 or 17, wherein the core of said matrix comprises alginate or hyaluronate, the containment layer comprises poly-L-lysine, and the outer layer comprises inorganic or organically modified inorganic sol-gel.

20. The method of claim 17, wherein the detectable signal is measured episodically, continuously, or programmed.

21. A biosensor comprising:
the multilayered or multicoated matrix of claim 1 with at least one binding protein specific for an analyte of interest embedded, entrapped or encapsulated therein; and
a transducing element.

22. A method of making a biosensor comprising the steps of:
(a) combining a binding protein with a reporter group attached thereto with an embedding, encapsulating or entrapping compound to form a core;
(b) contacting the core with a first coating solution for a sufficient time to coat or layer the core;
(c) isolating and drying the first coating solution to form a containment layer;
(d) contacting the containment layer with a second coating solution for a sufficient time to coat or layer the containment layer;
(e) isolating and drying the second coating solution to form a outer layer to obtain a biosensor.
